(19) United States
(12) Patent Application Publication
(10) Pub. No.: US 2005/0143694 A1
(43) Pub. Date: Jun. 30, 2005

(54) Wound care device

(76) Inventors: Nicolai Michael Schmidt, Naerum (DK); Truels Sterm Larsen, Frederiksberg (DK)

Correspondence Address: JACOBSON HOLMAN PLLC, 400 SEVENTH STREET N.W., SUITE 600, WASHINGTON, DC, 20004, US

(21) Appl. No.: 10/503,583

(22) PCT Filed: Feb. 14, 2003
(86) PCT No.: PCT/DK03/00100

(30) Foreign Application Priority Data
Feb. 15, 2002 (DK) PA 2002 00235

(51) Int. Cl. A61F 13/00

(52) U.S. Cl. 604/304

(57) ABSTRACT

A wound care device comprising foam composition wherein the retention factor Rv=R/Δ?V of the foam is at least 0.05 wherein R is the retention capacity (g/g) and Δ?V is the expansion of volume (% v/v) of the foam when wetted. A wound care device comprising foam with a high retention and low expansion of volume is achieved, which is desired in the treatment of wounds, especially chronic wounds.

### BACKGROUND OF THE INVENTION

1. Field of the Invention

The invention relates to a wound care device comprising foam.

Wound dressings with absorbent layers for absorbing body fluids are well known in the art. Absorbent layers are provided for the uptake of body fluids, especially wound exudates, so as to enable the wound dressing to keep a constant moist environment over the wound site, and at the same time avoiding maceration of the skin surrounding the wound.

Foam is often used as absorbent material for wound dressings in the treatment of exuding wounds, as it is capable of absorbing high amounts of exudates and feels soft and comfortable against the skin. However, the retention of foam is generally low, which may be a problem when used on a body part being exposed to pressure, such as pressure sores or foot ulcers. Using a dressing of low retention on such wounds may enhance the risk of maceration.

Foam absorbs exudate from the ulcer by the capillary effect accomplished by the cellular structure of the foam. As the foam absorbs, more and more cells will become filled with exudate. The foam matrix may have a more or less hydrophilic nature, depending of the properties of the used foam. A hydrophilic foam matrix will absorb and swell significantly when exposed to aqueous liquids such as wound exudate, while hydrophobic foam matrix will be substantially non-absorbent and thus have a low expansion of volume when wetted. Thus, absorbent foam will have cells filled with exudate and furthermore the matrix of the foam may swell to some extent due to an inherent partly hydrophilic nature of the foam.

2. Description of the Related Art

Foam used for wound care devices may be any suitable foam, being soft, skin-friendly and capable of handling exudate. Flexible polyurethane (PUR) foam is often used in wound care products due to its softness and skin-friendliness and good exudate absorption.

Most flexible PUR foams are of the poly-ether type, but any appropriate polyol may be used for preparation of PUR foam. Usually the poly-ether comprises ethylene-oxide (EO) and propylene-oxide (PO). Poly-etramethylene-oxide may be used instead of propylene-oxide. The hydrophilic properties are determined by the content of EO and PO. A hydrophilic foam contains more than 50% w/w of EO of the total poly-ether content and less than 50% w/w of PO of the total poly-ether content. For hydrophobic foam the content is less than 50% w/w of EO and more than 50% w/w of PO of the total poly-ether content.

Hydrophilic foam is usually preferred for use in wound dressings due to higher absorption and lower initial absorption time (IAT) compared to hydrophobic foam. However, a major disadvantage with this foam is the swelling of the foam when wetted by exudate, which may result in the foam being more than double the original size.

A moderate degree of swelling may be clinically acceptable and in rare cases even advantageous. However, a large degree of swelling may lead to different clinical problems. One problem is fixation of the dressing to the surface of the ulcerated site, which may eventually leading to leak and following maceration of the intact skin as well as general discomfort. Other serious problems are caused by the expansion accomplished by the nature of swelling. The expansion may result in an enhanced pressure to the wound or risk of wrinkling of the foam. The wrinkling may be so that the dressing folds and leaves pressure marks on the site of ulceration if it is pressurized. Another problem with swelling may be disintegration or delamination of the dressing, if the foam expands more than the other components of the dressing, the expansion may rupture the structure of the dressing.

Foam usually has a high absorption capacity but a low retention capacity. When mechanically applying pressure to the foam, the exudate in the cells of the foam will be forced out of the foam again and only the liquid inherently bound in the polymeric material of the foam matrix is retained.

However, high retention is often desired, especially in the treatment of pressure sores, where the dressing may be under pressure, in venous leg ulcers under compression treatment, and in diabetic foot ulcers where shoes or weight bearing may interfere.

The properties of relatively high absorption and relatively low retention is usually not desired in the treatment of exuding wounds, since it clinically may lead to maceration of the skin surrounding the wound. In order to avoid this, absorbent material with a high retention may be incorporated into the foam, such as absorbent material, often in the form of super absorbing particles (SAP) or super absorbent fibres (SAF) fixing the exudate in the foam.

Foams with incorporated super absorbent material are well known in the art, e.g. from European Patent No. 41 934 which discloses an open cell hydrophilic foam in which the absorbent material is incorporated in the cavities of the foam.

From DE Patent Application No. 43 28 190 is disclosed hydrophilic foam with super-absorbent particles incorporated therein. The foam provides a highly swelling soft matrix.

However, the above-mentioned references comprise polyurethane foam material with a primarily hydrophilic nature, and a high expansion of volume when wetted may be achieved.

In International Patent Application No. WO 01/60432 is disclosed an absorbent, substantially non-swellable PUR foam for a wound dressing. The foam has an expansion of volume of less than 10% v/v when wetted. The foam has some absorbent capacity but probably poor retention under pressure as the hydrophobic matrix does not absorb substantial amounts of exudate, and exposed to pressure the moisture will be pressed out of the cells, and may cause maceration of the wound and its surroundings.

U.S. Pat. Nos. 5,674,917 and 5,744,509 disclose PUR foam with high absorption and high retention, achieved by a high content of SAP. The foam is designed for use in diapers and sanitary napkins. The high content of SAP will inevitably give rise to a large expansion of the volume of the foam, rendering the foam unsuitable for use in wound care products.

In International Patent Application No. WO 01/15643 is disclosed an absorbent foam material. The foam is preferably a polysaccharide foam and may have a poor performance under pressure, such as a low elastic recovery due to the properties of the polysaccharide material.

European Patent Application No. 1 145 695 discloses an absorbent material comprising super absorbing polymers and fibres or foam, in a layered product. Examples show material comprising SAP entrapped in a fibrous material. The reference is silent with respect to properties of the foam mentioned.

International Patent Application No. WO 92/13576 discloses a hydrophilic foam impregnated with alginate. Due to the formulation of the foam a high expansion of volume would when wetted would be expected. Furthermore, by impregnating the foam with alginate, the alginate is not secured to the foam and may migrate out of the dressing and into the wound, which is highly undesired.

Thus, there is still a need for a wound care device comprising soft, skin-friendly foam, having a high retention capacity, but at the same time a relatively low expansion of volume when wetted. These properties are surprisingly achieved by the wound care device of the present invention.

### SUMMARY OF THE INVENTION

This invention relates to a wound care device comprising a foam composition.

### DETAILED DESCRIPTION OF THE PRESENT INVENTION

The invention relates to a wound care device comprising foam composition wherein the retention factor Rᵥ=R/ΔV of the foam is at least 0.05 wherein R is the retention capacity (g/g) and ΔV is the expansion of volume (% v/v) of the foam when wetted.

The retention factor Rᵥ describes the properties of the foam with respect to retention capacity and expansion of volume. It is desired to have foam with a high retention together with a low expansion of volume. A high value of Rᵥ indicates a high retention combined with low volume expansion, which is often desirable for wound care products.

In a preferred embodiment of the invention the retention factor Rv is at least 0.07 and more preferred at least 0.10.

The wound care device according to the invention is very suitable for treatment of chronic wounds due to the good retention, decreasing the risk of maceration and the low expansion of volume decreasing the risk of unintended pressure to the wound.

Furthermore, the wound care device according to the invention may easily be adapted to the wound site by cutting the foam into a desired shape and size.

The expansion of volume of the foam may be three-dimensional, i.e. expansions in three directions. A change of 100% v/v means then, that the size has doubled. Hydrophilic foam will often expand at least 100-300% v/v due to uptake of liquid in he polymeric matrix material, while hydrophobic foam may expand very little as the liquid will not be bound in the matrix material, but only trapped in the cells of the foam. Addition of absorbent material to the foam will usually increase the swelling even further.

A limited degree of volume change upon exudate uptake is desired, as a large volume change may give rise to folding and buckling of the foam as well as enhanced pressure against the wound site, which may cause discomfort for the user and even give rise to additional pressure sores.

Low expanding foam may have good volume efficiency i.e. that there is low degree of unused space in the dressing when absorbing and retaining exudate.

The device of the invention may preferably have a retention of at least 1 g/g, more preferred at least 1.5 g/g and most preferred 2 g/g, In one embodiment of the invention the foam has a retention capacity of at least 4 g/g more preferred 6 g/g and most preferred 8 g/g and an expansion of volume when wetted of less than 100% v/v more preferred less than 80% v/v and most preferred less than 60% v/v.

Preferably, the foam may have incorporated super-absorbent particles (SAP) or super-absorbent fibres (SAF).

The SAP may be incorporated into the foam in different ways, e.g. by mixing them into one or more of the components for preparation of the foam, or by impregnating or coating the foam. It is preferred that the SAP are incorporated during the preparation of the foam, as the SAP then will be fixed in the foam and migration of SAP into the wound is avoided. Furthermore, the SAP will be homogeneously distributed in the foam, which may be advantageous in order to prevent blocking of the foam.

The absorbent material of the particles may be any suitable material and may comprise super absorbent material, such as natural polysaccharides, carboxymethyl-cellulose (CMC), alginic acids, alginates, poly-acrylic acids, poly-acrylic amides, poly-acrylates, poly-methacrylates, poly-acrylonitrile, polyvinyl pyrrolidone, polyvinyl-lactams, polyvinyl pyridines, polyvinyl alcohol, polyvinyl acetate, gelatin or other hydrophilic polypeptides, carrageenans, pectin, xanthan, chitin, chitosan and salts, derivatives, copolymers and mixtures of the above type.

The foam may be any suitable foam composition for wound care devices, such as polyurethane, silicones, polyvinyl acetate, polyolefines or other suitable compositions.

Foam is a dispersion of air or other gases dispersed in a liquid or solid. Foam can be open cell or closed cell depending on whether the gas domains are interconnected or not. In a closed cell foam the gas is trapped in discrete cells whereas gas can move freely in an open cell foam. In the latter case the walls between the cells has been broken at some time of the foam production process. Foam of the present invention is based on the above definition and is only applying to solid materials.

Foam made of some materials such as polysaccharides has the disadvantage of not possessing adequate mechanical strength for some practical uses. For example, commercially available alginate dressings may collapse when exposed to pressure.

This inconvenience may be avoided by using a foam made from a polymeric material with elastomeric properties. Elastomers consist of three-dimensional network of covalently connected building blocks resulting in elongations of typically no less than their own length. Due to the elastic properties of the polymeric material, these foams usually have fine mechanical recovery.

The foam may preferably be poly-ether based polyurethane foam.

In one embodiment of the invention the foam may have a content of ethylene oxide of less than 50% w/w of the total poly-ether content.

In a preferred embodiment of the invention the foam may have a content of ethylene oxide of less than 40% w/w of the total poly-ether content.

In a more preferred embodiment of the invention the foam may have a content of ethylene oxide of less than 30% w/w of the total poly-ether content.

A fast initial absorption (low initial absorption time (IAT) value) is an advantage in a wound care device for exuding wounds.

Hydrophilic foams usually have a low IAT, while the hydrophobic foam may have a higher IAT.

In one embodiment of the invention the foam may have an initial absorption time (IAT) being less than 60 seconds, preferably less than 30 seconds, more preferred less than 20 seconds and most preferred less than 10 seconds.

A fast initial absorption time may be achieved by adding surface-active agents to the foam composition prior to or during mixing of the PUR components.

Surface-active agents are known in the art as surfactants. Surfactants are soluble compounds that help controlling surface tensions during foaming, but may as well contribute to the surface tension and surface hydrophilicity of the resulting foam. Surfactant properties are found in compounds containing hydrophilic chemical areas as well as hydrophobic areas i.e. anionic, cationic and nonionic surfactants. Examples of nonionic surfactants are EO/PO block-copolymers known as poloxamers, glycerol esters, and EO/PO siloxanes.

In a preferred embodiment of the invention the foam comprises EO/PO siloxanes.

When using traditional catalysts comprising organometallic compounds or amines such as dibutyltindilaurate, stannous-octoate and triethyldiamine, ethylmorpholine, 2,4,6-tris-dimethylaminomethyl-phenol etc., evaporation and migration may lead to environmental and human incompatibility problems.

In production of foam the inherent low vapour pressure of a catalyst and the reactivity in the cured product will be beneficial to lower the emission of the catalyst to the working environment as well as the environment in general.

This may be achieved by using chemically reactive catalysts.

In the final foam product the reactivity will stop the catalyst from migrating into the open ulcer via diffusion through the exudate. Thus, the risk of sensitisation, irritation and allergic reactions as well as harmful more long-term effects are minimized.

In one embodiment of the invention the foam comprises chemically reactive catalysts.

The catalyst may preferably be chosen from the group containing amines, such as tertiary amines or tertiary amines and hydroxyl groups.

Specially preferred catalysts may be dimethylethanolamine and NN-dimethylaminoethoxyethanol.

The foam composition of the device of the present invention provides foam with a surprisingly soft feel and fast initial exudate absorption i.e. low initial absorption time (IAT).

This may be obtained with the device of the present invention by combination of relatively hydrophobic poly-ether and use of various additives.

In a preferred embodiment of the invention the foam is poly-ether foam with less than 50% w/w EO of the total ether content and an inherent dimension change upon wetting of less than 20% v/v that can absorb exudate (IAT) within 30 seconds.

Preferably, the foam shows a high degree of elastic recovery both in wet and dry condition. It is desired that the foam rise again after being exposed to pressure, as well as it does not collapse when wetted.

Preferably, the device is in the form of a wound dressing, or a part of a wound dressing.

The dressing may be in the form of a single unit or a layered product.

The foam composition may in one embodiment constitute a dressing of the invention. In such case, the foam element may in itself show adhesive properties or it may not show adhesive properties and it will then typically be secured to the desired site using conventional means such as a cover dressing.

The dressing may comprise a skin-contacting surface comprising an area showing a skin friendly adhesive.

Such a dressing may suitably be a dressing comprising a substantially waterimpervious layer or film and a skin-friendly adhesive in which an absorbing foam composition according to the present invention is incorporated.

The skin-friendly adhesive may be any skin-friendly adhesive known per se, e.g. an adhesive comprising hydrocolloids or other moisture absorbing constituents such as the adhesives disclosed in U.S. Pat. No. 4,231,369 and in U.S. Pat. No. 4,367,732 comprising hydrocolloids.

A water impervious layer or film may be of any suitable material known per se for use in the preparation of wound dressings e.g. a foam, a non-woven layer or a polyurethane, polyethylene, polyester or polyamide film. A suitable material for use as a water impervious film is a polyurethane such as the low friction film material is disclosed in U.S. Pat. No. 5,643,187.

A dressing of the invention comprising a separate foam element is suitably located in the form of an “island” encircled by an adhesive border. The dressing may have any appropriate shape such as circular, oval, square or rectangular.

In another embodiment of the invention the device may be a wound cavity filler.

The cavity filler may e.g. be in the form of a foam element or a foam granulate or the foam may be combined with fibers, gel or hydrogel, or powder.

The device of the invention may comprise one or more active ingredients.

The device according to the invention may comprise deodorising agents.

The device may comprise one or more pharmaceutically or biologically active ingredients.

The device according to the invention may comprise wound healing associated indicator(s) such as indicators of pH, partial pressure of O₂, temperature, radical mechanisms or biotechnological assays, e.g. indicating formation of collagen.

This opens for a combined medical treatment of the wound and an easy and sterile application of the active ingredients, e.g. by incorporating active ingredients such as a cytochine such as growth hormone or a polypeptide growth factor giving rise to the incorporation of such active substances in a form being apt to local application in a wound in which the medicament may exercise its effect on the wound, other medicaments such as bacteriostatic or bactericidal compounds, e.g. iodine, iodopovidone complexes, chloramine, chlorohexidine, silver salts such as sulphadiazine, silver nitrate, silver acetate, silver lactate, silver sulphate, silver-sodium-thiosulphate, silver chloride or silver complexes, zinc or salts thereof, metronidazol, sulpha drugs, and penicillins, tissue-healing enhancing agents, e.g. RGD tripeptides and the like, proteins, amino acids such as taurine, vitamins such ascorbic acid, enzymes for cleansing of wounds, e.g. pepsin, trypsin and the like, proteinase inhibitors or metalloproteinase inhibitors such as Illostat or ethylene diamine tetraacetic acid, cytotoxic agents and proliferation inhibitors for use in for example surgical insertion of the product in cancer tissue and/or other therapeutic agents which optionally may be used for topical application, pain relieving agents such as lidocaine, chinchocaine or nonsteroid anti-inflammatory drugs (NSAIDS) such as ibuprofen, ketoprofen, fenoprofen or declofenac, emollients, retinoids or agents having a cooling effect which is also considered an aspect of the invention.

Material and Methods

Punching mould Ø 43 mm (Area 14.52 cm²)

Solution A: (8,298 g NaCl+0,368 g CaCl₂, 2H₂O per litre distilled water)

An incubator, 37° C., 50% relative humidity

A dial gauge for measuring by low force, on header with measuring table>50 mm and glass plate Ø 40 mm, thickness 1.9 mm

Calibrated steel ruler

Roller for pressing fluid out of sample consisting of two rolls Ø 60 mm where the weight of the top roll is 4000 gram.

Paper (Kleenex Medical Wipes 76:c 11*21 cm (x144=code 3020))

Analytical balance (accuracy 0.0001 g)

The retention and volume expansion of foam compositions was determined by the following test procedure:

Test Procedure:

A roundel (sample) with a diameter of 43,0 mm, D_{initial, was punched from the foam sheet to be tested. The roundel was weighted, W}ᵢₙᵢₜᵢₐₗ and the thickness, dᵢₙᵢₜᵢₐₗ, was measured. The roundel was placed in a petri dish and at least 50 ml solution A was added into the dish. The petris bulb with roundel and solution was left in incubator for 24 hours at 37° C. and 50% relative humidity. The roundel was then picked up with a pair of tweezers and was then weighted, w_{absorption} after having dripped for 10 seconds. Then the roundel was placed between two pieces of dry paper and rolled at the speed of 30 RPM though the plastic rollers. The rolling was repeated twice with new dry paper each time. In all, the roundel was rolled between dry paper tree times. The wet thickness, dᵣₑₜₑₙₜᵢₒₙ, and the wet diameter, Dᵣₑₜₑₙₜᵢₒₙ, were measured. The roundel was weighted again to obtain Wᵣₑₜₑₙₜᵢₒₙ.

Calculations:

The following results were calculated:

| | |
|---|---|
| Absorption, g/g: | A = (W_{absorption} − Wᵢₙᵢₜᵢₐₗ)/Wᵢₙᵢₜᵢₐₗ |
| Retention, g/g: | R = (Wᵣₑₜₑₙₜᵢₒₙ − Wᵢₙᵢₜᵢₐₗ)/Wᵢₙᵢₜᵢₐₗ |
| Volume | ΔV = ((Dᵣₑₜₑₙₜᵢₒₙ² * dᵣₑₜₑₙₜᵢₒₙ − |
| expansion, %: | Dᵢₙᵢₜᵢₐₗ² * dᵢₙᵢₜᵢₐₗ)/(Dᵢₙᵢₜᵢₐₗ² * dᵢₙᵢₜᵢₐₗ)) * 100% |

Determination of Initial Absorption Time

IAT (initial absorption time) was defined as the time (seconds) it takes for one drop (100 μl) of Solution A to wet the foam surface at ambient temperature.

Foam Preparation Procedure (Max. 60 g Portion):

Polyurethane foam sheets containing absorbing particles was prepared by the following procedure. The ingredients of the polyol phase and the absorbing particles were premixed with a standard laboratory mixer in a beaker. Then the isocyanate phase was added, and immediately after, the mixture was mixed again for 20 seconds forming a foaming emulsion. The emulsion was casted out between two siliconised polyethylene coated papers in a thickness of 2 mm on an electrically heated plate maintained at 50° C. Another electrically heated plate (weight: 890±10 g) maintained at 50° C. was placed on the top. The set up was left for 2 hours. The emulsion was turned into foam sheet as cross-linking and CO₂ formation occurred. The now formed foam sheet was removed from the plates and siliconised papers. Final strength was obtained after 2 days.

The formed foam sheet was then β-ray sterilized at 35 kGy in a single layer.

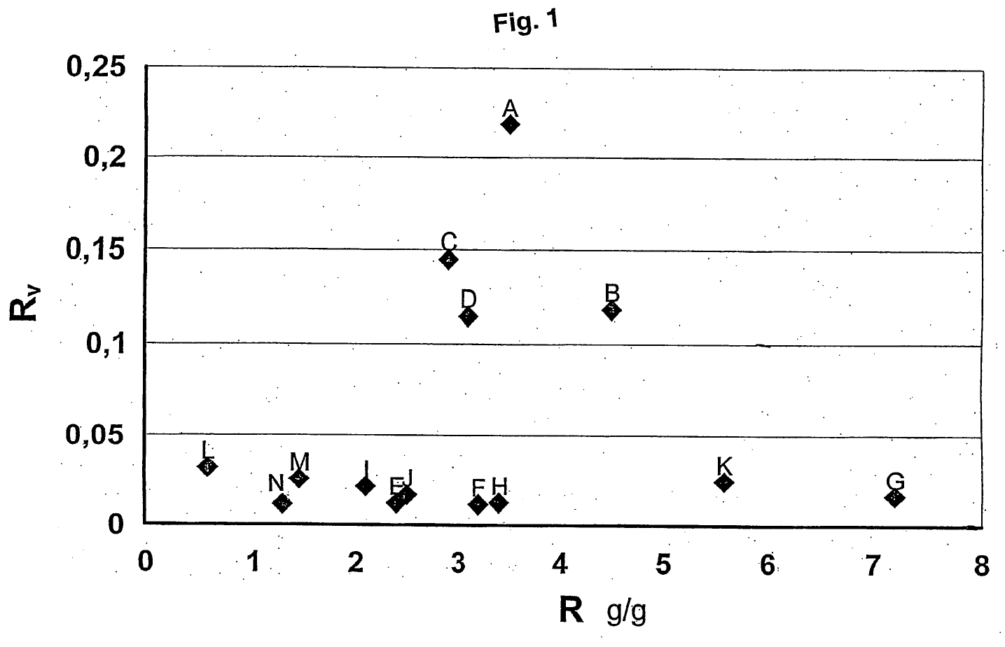

The following examples were prepared and retention and expansion was determined according to the above-mentioned procedure. The results are shown is Table 1 and FIG. 1.

### EXAMPLE A

A foam sheet containing 15% particles was prepared using the “Foam preparation procedure” with the following ingredients:

Polyol phase:
30.00 g Lupranol 2042 (BASF)
0.36 g distilled water
0.30 g Polycat 17 (AirProducts)
0.20 g Silpur 9000 (GE Bayer Silicones)

Super absorbing particles:
7.45 g Norsocryl S35 (Atofina)

Isocyanate phase:
11.30 g Lupranat MP 102 (BASF)

### EXAMPLE B

A foam sheet containing 25% particles was prepared using the “Foam preparation procedure” with the following ingredients:

Polyol Phase:
30.00 g Lupranol 2042 (BASF)
0.36 g distilled water
0.30 g Polycat 17 (AirProducts)
0.20 g Silpur 9000 (GE Bayer Silicones)

Super absorbing particles:
14.05 g Norsocryl S35 (Atofina)

Isocyanate phase:
11.30 g Lupranat MP 102 (BASF)

### EXAMPLE C

A foam sheet containing 15% particles was prepared using the “Foam preparation procedure” with the following ingredients:

Polyol phase:
27.00 g Lupranol 2042 (BASF)
3.00 g Voranol CP 1421 (DOW)
0.36 g distilled water
0.30 g Polycat 17 (AirProducts)
0.20 g Silpur 9000 (GE Bayer silicones)

Super absorbing particles:
7.45 g ASAP 2300 (BASF plc)

Isocyanate phase:
11.40 g Lupranat MP 102 (BASF)

### EXAMPLE D

A foam sheet containing 25% particles was prepared using the “Foam preparation procedure” with the following ingredients:

Polyol phase:
27.00 g Lupranol 2042 (BASF)
3.00 g Voranol CP 1421 (DOW)
0.36 g distilled water
0.30 g Polycat 17 (AirProducts)
0.20 g Silpur 9000 (GE Bayer Silicones)

Super absorbing particles:
14.05 g ASAP 2300 (BASF plc)

Isocyanate phase:
11.40 g Lupranat MP 102 (BASF)

### EXAMPLE E-N

Samples of known foam wound care products were analysed according to the above “Test procedure”. Example E was Trufoam from Maersk Medical, Example F was Tielle from Johnson & Johnson, Example G was Cutinova Cavity from Beiersdorf, Example H was Tielle Packing from Johnson & Johnson, Example I was PolyWic from Ferris, Example J was Biatain from Coloplast, Example K was Cutinova Foam from Beiersdorf, Example L was Lyofoam from Seton Health Care Group plc, Example M was Allevyn from Smith & Nephew and Example N was Mepilex from Moelnlycke. The results are shown in Table 1 and FIG. 1.

TABLE 1

| No. | Product | ΔV (% v/v) | R (g/g) | Rᵥ |
|---|---|---|---|---|
| A | Example A | 16 | 3.5 | 0.219 |
| B | Example B | 38 | 4.5 | 0.118 |
| C | Example C | 20 | 2.9 | 0.145 |
| D | Example D | 27 | 3.1 | 0.115 |
| E | Trufoam | 199 | 2.4 | 0.012 |
| F | Tielle | 280 | 3.2 | 0.011 |
| G | Cutinova Cavity | 436 | 7.2 | 0.017 |
| H | Tielle Packing | 273 | 3.4 | 0.012 |
| I | PolyWic | 98 | 2.1 | 0.021 |
| J | Biatain | 150 | 2.5 | 0.017 |
| K | Cutinova Foam | 228 | 5.6 | 0.025 |
| L | Lyofoam | 18 | 0.6 | 0.032 |
| M | Allevyn | 57 | 1.5 | 0.026 |
| N | Mepilex | 114 | 1.3 | 0.011 |

FIG. 1 shows the retention factor Rv as a function of retention R. As can be seen from the figure, the examples A-D of the invention clearly differs from the known products of the wound care business by their low expansion combined with a high retention. The known products, Example E-N shows Rᵥ well below 0.05.

1. A wound care device comprising foam composition wherein the retention factor Rᵥ=R/ΔV of the foam is at least 0.05 wherein R is the retention capacity (g/g) and ΔV is the expansion of volume (% v/v) of the foam when wetted.

2. A device according to claim 1 characterised in that the retention factor Rᵥ of the foam is at least 0.07.

3. A device according to claim 1 characterised in that the foam has incorporated super-absorbent particles or fibres.

4. A device according to claim 1 characterised in that the device is a wound dressing.

5. A device according to claim 1 characterised in that foam is polyurethane foam.

6. A device according to claim 5 characterised in that the foam comprises poly-ether and has a content of ethylene oxide of less than 50% of the total content of polyether.

7. A device according to claim 5 characterised in that the foam has a content of ethylene oxide is less than 40% of the total poly-ether content.

8. A device according to claim 1 characterised in that the foam has an initial absorption time is less than 10 seconds.

9. A device according to claim 1 characterised in that the foam comprises EO/PE siloxanes.

10. A device according to claim 1 characterised in that the device comprises one or more active ingredients.
